(19) United States
(12) Patent Application Publication
(10) Pub. No.: US 2009/0022799 A1
(43) Pub. Date: Jan. 22, 2009

(54) COMPOSITIONS THAT CONTAIN BETA-GLUCAN TO BE USED FOR THE PREVENTION AND TREATMENT OF DISEASE AND METHODS FOR THEIR USE

(76) Inventor: Shikha Pramanik Barman, Bedford, MA (US)

Correspondence Address: CAMERON INTERNATIONAL CORPORATION, P.O. BOX 1212, HOUSTON, TX, 77251-1212, US

(21) Appl. No.: 11/867,309

(22) Filed: Oct. 4, 2007

Related U.S. Application Data
(60) Provisional application No. 60/850,284, filed on Oct. 6, 2006.

(51) Int. Cl. A61K 9/28; A61K 31/715; A61K 47/06; A61P 35/00; A61P 37/02; A61P 25/28; A61P 25/16; A61P 15/02; A61P 1/00

(52) U.S. Cl. 424/474; 514/54; 424/278.1

(57) ABSTRACT

This application for patent discloses β-Glucan containing compositions, dosage forms, delivery methods, and techniques for the purposes of preventing and treating mucositis through wound healing and tissue re-organization, as well as the treatment of immune disorders such as Alzheimer's disease, Multiple Sclerosis and Parkinson's disease. β-Glucan containing compositions, dosage forms and methods to deliver therapeutic and prophylactic vaccines and anti-cancer drugs are also disclosed, as are methods to stabilize a drug to enhance its shelf life and to enhance the level and duration of its bioactivity.

### FIELD OF THE INVENTION

This invention describes the utility of β-Glucan added to drug-containing compositions as it relates to the delivery to bioactive compounds such as biologics and drugs, for the purpose of drug stabilization, wound healing, vaccinating and drug therapy for alleviation of clinical disease.

### BACKGROUND OF THE INVENTION

### Beta Glucan

Beta-(1→3)-d-Glucan is an integral cell wall component of a variety of fungi, plants, and bacteria. β-Glucan is an immune system modulating compound that has been demonstrated to act as a non-specific macrophage activator. A glucan is a polymeric glucose (a polysaccharide), naturally occurring as poly-branched beta-1,3-D-glucan found in a variety of fungal cells (FIG. 1). Poly-branched beta-1,3-D-glucan is a naturally occurring polysaccharide that can be found in a variety of fungal cells including cell walls of yeast, Saccharomyces cerevisiae. As any other glucan (or polyglucose), it consists of glucose units linked together.

Out of different glucans, the beta-1,3-D-glucan configuration has been shown to act as a non-specific immune-activator. A specific receptor has been identified on the cells of macrophage origin that binds to the beta-1,3-D-glucan molecule. This receptor is a protein complex that appears to be present throughout the whole differentiation cycle of macrophages, starting in the bone marrow. Mature macrophages are found in virtually all the tissues including the central nervous system. When a macrophage encounters beta-1,3-D-glucan, it becomes activated. All immune-modulating functions, including phagocytosis (ability to engulf foreign cells and particles), release of certain cytokines (intercellular hormones), and the processing of antigens are enhanced in the presence of β-Glucan. A linear β-D(1,3)-linked glucopyranose polymer with a triple-helical conformation, it is produced by an adapted strain of Euglena gracilis. In preclinical studies, algal glucan has been intravenously administered at doses up to 25 mg/kg body weight and was well tolerated. Human clinical trials have indicated that β-Glucan is safe and well-tolerated. It has been used as an adjuvant for enhancement of both humoral and cell-mediated immunity¹. It is approved by the FDA as Zymosan®. It is a white, odorless, crystalline material. It is insoluble in water and forms a suspension in aqueous solution. Median particle size for β-Glucan particles is 3.7-4.6 μm. ¹ Underhill, et, al. 1999. “The toll-like receptor 2 is recruited to macrophage phagosomes and discriminates between pathogens. Nature 401:411; Young, S.-H. et. al. 2001. “Molecular Mechanism of Tumor Necrosis-α Production in 1→3 β-Glucan-Activated Macrohages, J. Biol. Chem. 276: 20781

### DISCUSSION OF THE PRIOR ART

U.S. Pat. No. 4,138,479 discloses a water soluble immunopotentiating agent derived from yeast cell wall material, including mechanically disrupted yeast cell walls, proteolyzed yeast cell wall material and carbohydrate-protein complexes found in yeast cell wall material, through extraction with a water-phenol mixture. The water soluble agent is isolated from the water phase and can be further purified, as through dialysis, to remover low molecular weight components.

U.S. Pat. No. 4,182,751 pertains to a medicament which stimulates non-specific immunity and contains a phenol-soluble extract of micro-organisms, wherein the phenol-soluble extract or fraction thereof of bacteria, yeasts and/or protozoa is rendered water soluble and free from endotoxin, substantially free from phenol and used in a portion for stimulating the receiving organism.

U.S. Pat. No. 4,337,243 pertains to a medicament which stimulates non-specific immunity and contains a phenol-soluble extract of micro-organisms, wherein the phenol-soluble extract or fraction thereof of bacteria, yeasts and/or protozoa is rendered water soluble and free from endotoxin, substantially free from phenol and used in a portion for stimulating the receiving organism.

U.S. Pat. No. 4,695,549 pertains to a process for obtaining a sterile, apyrogenic product for promoting oxidative phosphorylation and suitable for therapeutic or cosmetic compositions, starting from yeast, in which any type of yeast is subjected to a process of plasmolysis, followed by treatment with proteolytic enzymes and then with diamine oxidase, after which the proteins present in the solution are precipitated by alcohols, the solution pH is stabilized, and the solution concentrated at low temperature under vacuum.

U.S. Pat. No. 4,739,046 describes a class of soluble phosphorylated glucans and the process for making the same. According to a preferred embodiment, the soluble phosphorylated glucan is derived from the yeast Saccharomyces cerevisiae. The soluble phosphorylated glucans are useful for prophylactic and therapeutic applications against neoplastic, bacteria, viral, fungal and parasitic diseases. Additionally, they may be administered as a non-toxic adjuvant, in combination with chemotherapy. The soluble phosphorylated glucans are also useful for stimulating macrophage cells, either in vivo or in vitro, to produce a cytotoxic/cyctostatic factor effective against cancer cells.

U.S. Pat. No. 4,761,402 describes a class of soluble phosphorylated glucans and the process for making the same. According to one embodiment, the soluble phosphorylated glucan is derived from the yeast Saccharomyces cerevisiae. The soluble phosphorylated glucans are useful for prophylactic and therapeutic applications against neoplastic, bacteria, viral, fungal and parasitic diseases. The soluble phosphorylated glucans are used either alone or in combination with a known antimicrobial agent for prophylactic and therapeutic antimicrobial applications. Additionally, they may be administered as a non-toxic adjuvant, in combination with chemotherapy. The soluble phosphorylated glucans are also useful for stimulating macrophage cells, either in vivo or in vitro, to produce a cytotoxic/cyctostatic factor effective against cancer cells.

U.S. Pat. No. 4,818,752 describes a class of soluble phosphorylated glucans and the process for making the same. According to one embodiment, the soluble phosphorylated glucan is derived from the yeast Saccharomyces cerevisiae. The soluble phosphorylated glucans are useful for prophylactic and therapeutic applications against neoplastic, bacteria, viral, fungal and parasitic diseases. The soluble phosphorylated glucans are used either alone or in combination with a known antimicrobial agent for prophylactic and therapeutic antimicrobial applications. Additionally, they may be administered either alone or as a non-toxic adjuvant, in combination with chemotherapy. The soluble phosphorylated glucans are also useful for stimulating macrophage cells, either in vivo or in vitro, to produce a cytotoxic/cyctostatic factor effective against cancer cells.

U.S. Pat. No. 4,833,131 describes a class of soluble phosphorylated glucans and the process for making the same. According to one embodiment, the soluble phosphorylated glucan is derived from the Yeast Saccharomyces cerevisiae. The soluble phosphorylated glucans are useful for promoting the wound healing process. The soluble phosphorylated glucans are also useful for prophylactic and therapeutic applications against neoplastic, bacteria, viral, fungal and parasitic diseases. The soluble phosphorylated glucans are used either alone or in combination with a known antimicrobial agent for prophylactic and therapeutic antimicrobial applications. Additionally, they may be administered either alone or as a non-toxic adjuvant, in combination with chemotherapy. The soluble phosphorylated glucans are also useful for stimulating macrophage cells, either in vivo or in vitro, to produce a cytotoxic/cyctostatic factor effective against cancer cells.

U.S. Pat. No. 4,992,540 describes three dimensional glucan matrix compositions prepared by separating growing yeast from its growth medium, subjecting the yeast with cell walls intact to an alkali material, thereby extracting whole glucan particles having an intact cell wall structure. The whole glucans can then, optionally, be treated with acetic acid to alter the .beta.(1-6) linkages, or with glucanase to alter the .beta.(1-3) linkages. The glucans have viscosity characteristics dependent upon the strain of yeast utilized and are useful as stabilizers or thickeners.

U.S. Pat. No. 5,019,391 describes a composition for the treatment of the skin comprising a fraction of a mechanically obtained lysate of yeast cultures of the species Saccharomyces cerevisiae. The translation system contained in the compositions of the invention are obtained by lysing cultures of Saccharomyces cerevisiae. The application of such composition, in any suitable form, such as a cream, ointment, gel or the like, to skin promotes protein biosynthesis by the skin cells so that the metabolism of the extracellular matrix of the skin is restored to the physiologically correct balance and the skin is revitalized.

U.S. Pat. No. 5,037,972 describes three dimensional glucan matrix compositions that are prepared by separating growing yeast from its growth medium, subjecting the yeast with cell walls intact to an alkali material, thereby extracting whole glucan particles having an intact cell wall structure. The whole glucans can then, optionally, be treated with acetic acid to alter the .beta.(1-6) linkages, or with glucanase to alter the .beta.(1-3) linkages. The glucans have viscosity characteristics dependent upon the strain of yeast utilized and are useful as stabilizers or thickeners.

U.S. Pat. No. 5,082,936 describes three dimensional glucan matrix compositions that are prepared by separating growing yeast from its growth medium, subjecting the yeast with cell walls intact to an alkali material, thereby extracting whole glucan particles having an intact cell wall structure. The whole glucans can then, optionally, be treated with acetic acid to alter the .beta.(1-6) linkages, or with glucanase to alter the .beta.(1-3) linkages. The glucans have viscosity characteristics dependent upon the strain of yeast utilized and are useful as stabilizers or thickeners.

U.S. Pat. No. 5,519,009 describes a sorbitol-solubilized yeast glucan, its method of preparation, and methods of use.

U.S. Pat. No. 5,573,785 describes a cosmetic component produced by dispersing in water a water-soluble fiber composed of about 4 to 6 weight percent beta-glucan, about 1 to 5 weight percent fat, about 80 to 94 weight percent carbohydrates and less than 8 weight percent protein.

U.S. Pat. No. 5,576,015 describes substantially purified beta (1,3) glucan extracts obtained from yeast cell walls, particularly finely ground, and nutritional and dermatological applications of same.

U.S. Pat. No. 5,786,343 describes a phagocytosis-stimulating composition comprising, and preferably consists essentially of (a) a phagocytosis-stimulating substance, (b) ascorbic acid or a derivative thereof, and (c) a pharmaceutically acceptable carrier. The phagocytosis-stimulating substance suitably can be a yeast cell wall extract, such as beta-(1,3)-D-glucan.

### SUMMARY OF THE INVENTION

This invention discloses β-Glucan containing compositions and dosage forms, delivery methods and techniques for the purposes of treating mucositis through wound healing and tissue re-organization, treatment of immune disorders such as Alzheimer's disease, Multiple Sclerosis and Parkinson's disease. Beta-Glucan containing compositions, dosage forms and methods to deliver therapeutic and prophylactic vaccines are also disclosed. Furthermore, this invention discloses methods to stabilize a drug to enhance its shelf life and to enhance the level and duration of its bioactivity.

### BRIEF DESCRIPTION OF THE DRAWINGS

FIG. 1: Method of Preparation of B-Glucan Nanoparticles

FIG. 2: Preparation of cationic β-Glucan nanoparticles

FIG. 3: Method of Drug Encapsulation in β-Glucan Containing Polymer

### DETAILED DESCRIPTION OF THE INVENTION

### 01 Scope of the Invention

This invention describes optimally designed route-specific β-Glucan containing compositions which can be used to:
(a) deliver immune-modulating drugs for the therapy of immune-related diseases (such as multiple sclerosis, Alzheimer's disease, Parkinson's disease) or co-deliver with prophylactic vaccines (influenza, childhood vaccines, bird flu, West Nile virus, meningitis) or therapeutic vaccines (HPV, cancer, Alzheimer's Disease) to enhance their immunity-generating capability.
(b) deliver a biocompatible, thermo-stable, polymeric matrix (with or without drug) to mucosal tissue to prevent or treat diseases that are caused by destruction of the mucosa due to a compromised immune system (mucositis caused chemotherapy, AIDS, radiation treatment, prolonged antibiotic treatment).
   The delivery system described in this invention, will be comprised of β-Glucan and/or its derivatives thereof, a biocompatible matrix that by virtue of its own functionalities and incorporated ingredients render the composition appropriate for the route of delivery and improves its efficacy and overall functionality, and optionally a drug.
   By “route-of-delivery”, it is meant that the compositions described in this invention can be delivered by the oral route, nasal route, ocular route, rectal route, vaginal route, pulmonary route and dermal route.

By “β-Glucan”, it is meant that β-Glucan in any form is applicable, such as particulate β-Glucan, or soluble β-Glucan or insoluble β-Glucan.

By particulate, it is meant that micro-particles and nano-particles of β-Glucan are applicable. The size of the microparticulate β-Glucan can be between 1 micron and 100 microns. The size of the nanoparticulate β-Glucan can be between 10 nm to 1000 nm.

### 02 Concept 1

### Delivery Systems and Dosage Forms to Deliver Vaccines or Immune Modulating Drugs

The drug delivery compositions in the form of a pre-formed, fast-disintegrating dry film, can be used to deliver vaccines or immunomodulatory compounds via sublingual, oral, dermal, and ocular routes. Furthermore, the delivery dosage form, by virtue of its physical state, can have a chemically stabilizing effect to the incorporated drug by holding the bioactive relatively immobile to molecular motion.

In a preferred embodiment, compositions comprised of a water-soluble film-forming polymer, a water-soluble bioadhesive polymer, β-Glucan, a therapeutic or a prophylactic drug and other components appropriate for enhancement of the overall functionality of the drug delivery system, will be pre-formed into a pliable film that can be applied to tissues of the oral cavity, preferably to the very hydrated tissues of the sublingual and buccal space.

A dry film applied to moist tissues will re-hydrate rapidly and dissolve within a few minutes, preferably in less than 5 minutes. The hydrated film will adhere to the mucosal surface due to its bioadhesive characteristics. Likewise, the dry film can be applied to a portion of the ocular epithelium, to deliver a drug to treat diseases of the eye.

### A. Preparation of the Dry Film

To form the “dry film”, a film-forming polymer or a combination of polymers such as poly(vinyl pyrrolidone) (PVP), polyethylene glycol (PEG), hydroxypropyl methyl cellulose (HPMC), poly(vinyl alcohol), poly(vinyl acetate) and derivatives thereof, will be dissolved in a solvent between 1-10% w/v. The solvent used will be ethanol, or a mixture of ethanol (50-75% v/v) and water (50-25% v/v). The drug, a bioadhesive polymer (such as hyaluronic acid, xanthan gum, alginate) and the β-Glucan will be incorporated into the polymer-containing solvent system. The incorporated β-Glucan can be particulate, preferably nano-dispersed into the solvent with a dispersing agent. The drug/β-Glucan/polymer/solvent mixture is cast on a glass plate and then gently evaporated in a chamber under a nitrogen flow and then dried under gentle vacuum between 5-10 hours at room temperature. The film after drying should be pliable and integral. Films prepared in this process generally will dissolve within 5 minutes in water and 0.9% saline.

### B. Preparation of Fast-Disintegrating Tablets

Fast Disintegrating tablets can be prepared in the following method: B-Glucan (0.1-1% w/w), Drug (as required, to 20 mg), HPMC (10%), Magnesium Stearate (0.5%), talc (1%), Sodium Starch Glycolate (15%), gelatinized starch (10%) was mixed in a V-Blender at room temperature for 4 hours. The resultant blend was formed into 50 mg tablets by direct compression of the blend, by a tablet press.

The tablets have a hardness of 10 Kiloponds and a disintegration time of 5 minutes.

### C. Biocompatibility

All components of the therapeutic “dry film” composition will be biocompatible with tissue.

By “biocompatible”, it is meant that the components of the delivery system will not cause tissue injury or injury to the human biological system. To impart biocompatibility, polymers and excipients that have had history of safe use in humans or with GRAS (Generally Accepted As Safe) status, will be used preferentially. By biocompatibility, it is meant that the ingredients and excipients used in the composition will ultimately be “bioabsorbed” or cleared by the body with no adverse effects to the body. For a composition to be biocompatible, and be regarded as non-toxic, it must not cause toxicity to cells. The composition developed in this invention, uses excipients and ingredients only in quantities that are considered safe by regulatory health and medicine authorities. Use of biocompatible ingredients is a necessity for the applications described herein, due to the immune-compromised nature of the persons being treated, as in cancer patients or patients with multiple sclerosis.

### D. Thermal Stabilization

The formulation of all components into a dry film or a pressed tablet has the added advantage of stabilizing the drug delivery system, to enable storage at room temperature since the system is in a dried physical state. The advantage of stabilizing bioactives such as protein-based vaccines is that this provides ease of transport to hot, arid parts of the world with no refrigeration constraints. As an example, the cold transport of heat-sensitive vaccines to rural parts of the world is challenging and difficult, due to the lack of infrastructure in these areas. Thus, a dosage form that is a dried film is stable at ambient temperatures, is applied with ease without the use of needles, and can be carried by health care workers in ambient temperature transport packs, is desirable and satisfies an unmet clinical need.

As stabilizing agents, excipients such as disaccharides such as sucrose and trehalose, sorbitol, cyclodextrins and its derivatives thereof, or other polymers such as polyethylene glycols or cellulosics, may be added to the composition.

Charged polymers such as lecithin, albumin, chitosan, hyaluronic acid, poly(amino acids), PEI, PAMAM, PEG-PEI, PEG-PAMAM and other molecules that are charged (positive, negative and zwitterionic) such as cholesteryl sulfate, protamine sulfate, glycocholic acid, deoxycholic acid, cholic acid, gluconic acid, glutamic acid, taurocholic acid may be added to the composition to enable further stabilization of the drug by forming a charged complex with the drug. For example, a positively charged molecule such as DNA or a nucleic acid can be stabilized by interaction with a charged polymer such as albumin, lecithin, poly(lysine) or poly(ethylene-imine) (PEI) or PAMAM or a charged molecule such as CTAB, protamine, etc.

Techniques such as annealing of films has been shown to impart stability to incorporated drug by the alignment of the polymer chains and can be used in this application to enhance stability. Other techniques used to align the polymer chains to impart stability may be cold drawing or compression or spin coating.

In another example of a heat-stable dosage form, the matrix of the film may have liquid crystalline properties. A liquid crystal is defined as a physical state that has the characteristics of solid and liquid states. That is, a liquid crystal has flow properties characteristic of a liquid and has the lattice structure characteristic of a crystalline solid. Examples of liquid crystalline substances are cholesterol and cholesterol derivatives, hydroxyl-propyl chitosan, cellulose derivatives, etc.

### D. Other Dosage Forms

A fast dissolving dosage form such as a tablet, troche, or lozenge, or a dry film is preferable for delivery of the β-Glucan containing prophylactic or therapeutic in the oral space.

The dry film, comprised of all its therapeutic constituents may be prepared by spin-coating, casting or film-extrusion.

In a preferred embodiment, the β-Glucan incorporated in the “dried film” dosage form will be in the form particles, micron sized and nano-sized, charged or uncharged.

A “fluid” dosage form is understood to mean a non-viscous liquid, a viscous liquid, a soft gel, or a cream as appropriate for the given application.

### Preparation of β-Glucan Nanoparticles

### Methods

### Ultrasonication and Spray-Drying

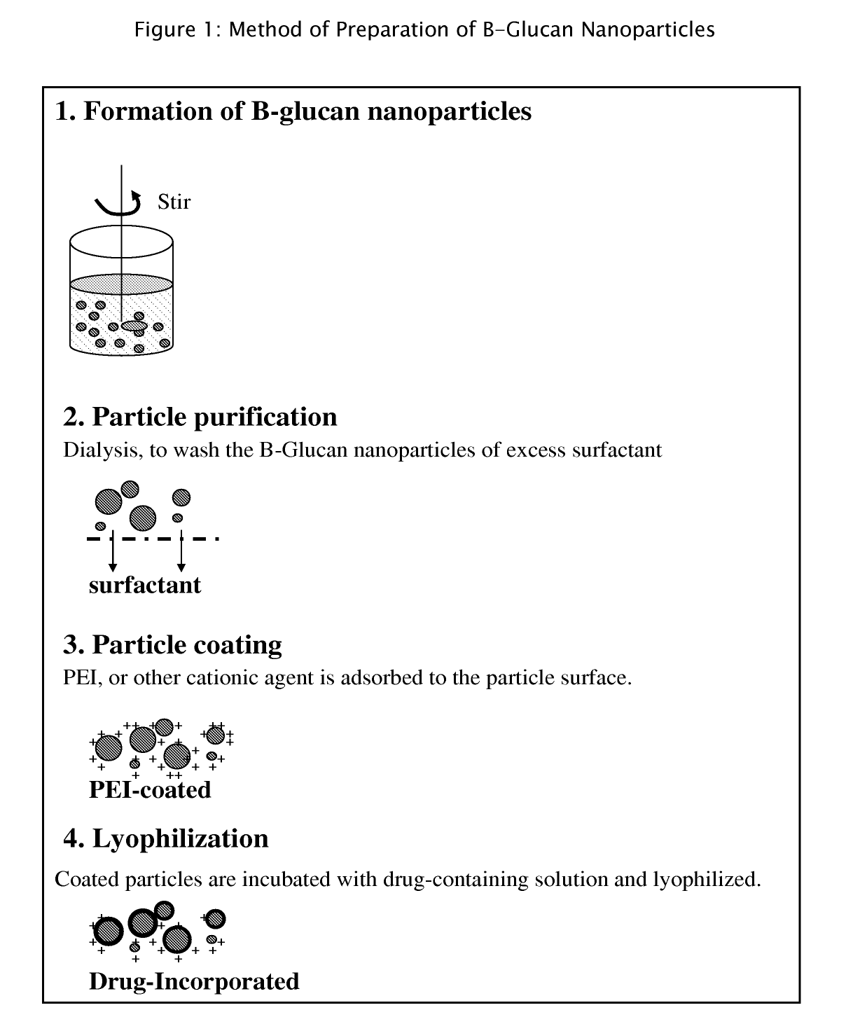

1 g of β-Glucan was probe sonicated using a Sonicator 300 purchased from Misonix, Inc., in 100 ml of water containing 1% Tween 20, 0.01% sodium deoxycholate for 5 minutes, in a 250 ml polypropylene centrifuge tube. The centrifuge tube containing the dispersion was cooled with an ice bath during the sonication process. The probe was held at mid-length in the liquid. The dispersion was then spray-dried as shown in FIG. 1. The dispersion was tested for homogeneity and particle size distribution using a Malvern particle size analyzer.

The particles were homogenous, with a mean diameter of 500-550 nm.

Preparation of β-Glucan Nanoparticles Coated with a Cationic Polymer, Polyethyleneime (PEI)

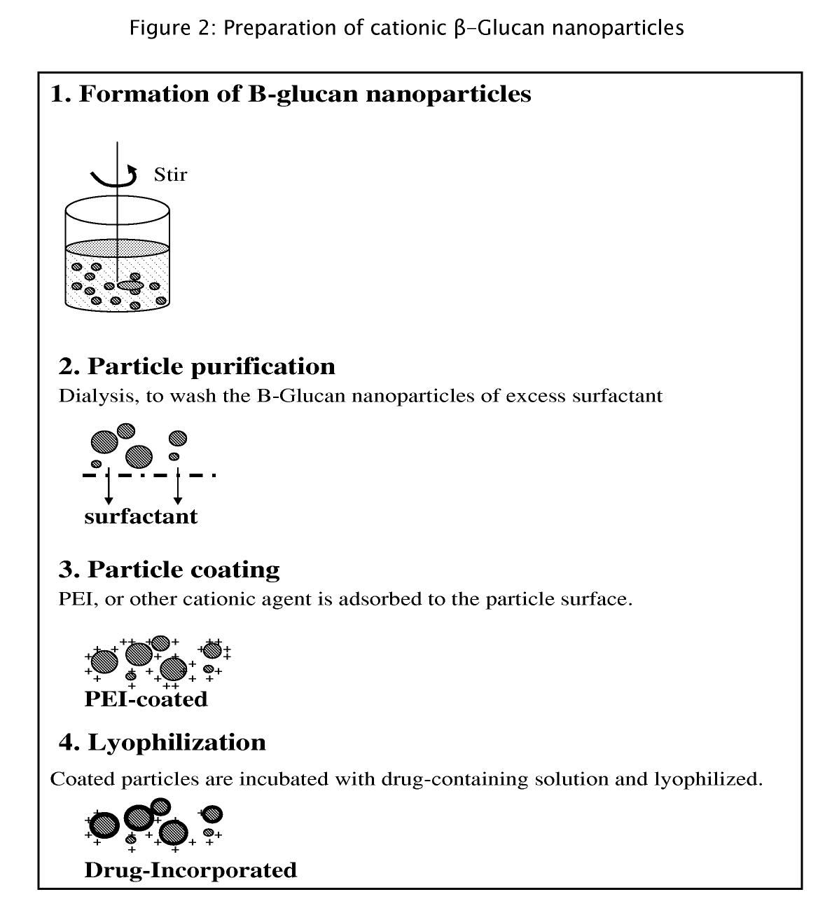

B-Glucan nanoparticles were prepared as described in the previous example. A solution of polyethyleneimine (PEI) was prepared in water at a concentration of 1% w/v (See FIG. 2). The solution of polyethyleneimine also contained 0.1% Tween 20. The dispersion of β-Glucan nanoparticles were centrifuged at 4500 RPM for 20 minutes at room temperature and the supernatant discarded. The pellet of nanoparticles was re-suspended with the solution of PEI and rotated on a rotating tumbler for approximately 2 hours. This step incorporates the cationic polymer onto the negatively charged B-glucan polymer. The resultant dispersion was washed by dialysis in water for injection containing 0.5% Tween 20. The particles were characterized for particle size distribution by a Malvern particle size analyzer and net positive charge measured using a zeta potentiometer. The particles were then incorporated mixed in with an aqueous solution containing drug and lyophilized. Results: The particles were well-suspended and the dispersion stable. The particle size measured approximately 300-800 nm with a zeta potential of +16.

### Preparation of PLG-Encapsulated β-Glucan (+Drug) Microparticles

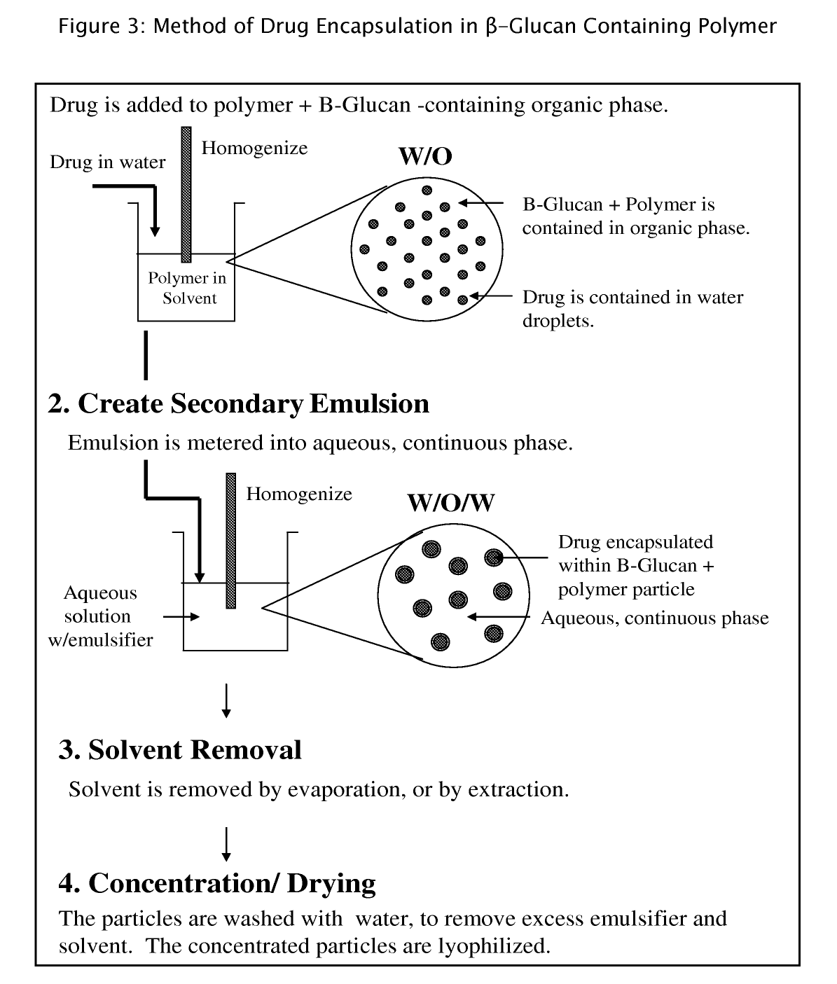

10 mg of β-Glucan and 100 mg poly(lactide-co-glycolide) (PLG) was dissolved in 2 ml of methylene chloride in a 15 ml centrifuge tube (See FIG. 3). The solution was homogenized for 5 minutes using a Silverson Homogenizer, then slowly poured into a solution containing 1% polyvinyl alcohol. The resultant milky dispersion was stirred at room temperature for a half hour, then centrifuged at 4500 RPM for 10 minutes. The supernatant was discarded and the microparticles resuspended using water for injection (WFI). The resuspended microparticles were shaken for a half hour on a roller-shaker, then centrifuged at 4500 RPM for another 10 minutes. The supernatant was discarded again, the microparticles resuspended again in water. This washing process was repeated for a total of three times. The washed and concentrated microparticles were dried by lyophilization.

The size of the nanoparticles will be less than 1 micron in size in average. Particles less than 10 microns are subject to phagocytosis and those less than 1 micron can be taken up by the mucosa and activate the immunomodulatory cells. For vaccine delivery or for delivery of an immunotherapeutic, co-delivery of nanoparticulate β-Glucan as an adjuvant to the buccal and sublingual tissue is an excellent alternative to vaccines delivered by injection.

The size of the microparticles will be between 1-100 micron in diameter. Particles less than 10 microns will be taken up by cellular phagocytosis. Particles greater than 10 microns will perform as sustained delivery drug depots to deliver over a prolonged period. By “prolonged”, it means delivery of the bioactive agent for a time frame greater than 30 minutes.

In another embodiment, the β-Glucan incorporated in the dosage form will be soluble in water.

In another embodiment, the β-Glucan incorporated in the dosage form will be insoluble in water.

In another embodiment, the β-Glucan incorporated in the dosage form will be amphiphilic in nature, that is both hydrophilic and hydrophobic.

In one embodiment of the invention, the matrix may comprise of ingredients that render further immune modulation. Examples of co-adjuvants may be in the form of oil-in-water microemulsions, synthetic microparticles, mineral salts, aluminum hydroxide (Alum, or Alhydrogel), CPG-nucleotides, peptidoglycans, arabinomannan, glycolipids, etc. Other ingredients that may be contained in the matrix may add other functionalities, such as cell attachment, mucoadhesivity, etc.

In a further enhancement, the therapeutic composition may include a penetration enhancer which aids rapid transport of the pharmaceutical substance across the mucosal epithelium. The therapeutic composition can also include other components that are compatible with the pharmaceutical substance and the biocompatible polymer. Examples of penetration enhancers include, but are not limited to triacetin, menthol, eucaplyptol, benzyl alcohol, deoxycholate, polyethylene glycol, polypropylene glycol, tocopherol and PEGylated tocopherol.

In a preferred embodiment of this invention, the β-Glucan nanoparticles co-delivered with the bioactive drug is coated with a bioadhesive molecule or polymer to enhance the residence time of the nanoparticles at the site, until their uptake by the cells present in the epithelial lining of the oral mucosa. Given that β-Glucan has been demonstrated to attach to cells prior to cellular uptake, it is entirely rational to incorporate bioadhesive-coated β-Glucan nanoparticles into the oral film dosage form containing an immunotherapeutic or a prophylactic immunomodulatory drug such as a vaccine. Nanoparticles have a higher rate of cellular uptake than microparticles. Thus, it is rational that the β-Glucan particles be nano-sized for this application.

In an embodiment of the invention, the fast dissolving dosage form will contain a bioadhesive polymer, to increase the residence time of the formulation in the oral cavity. Examples of bioadhesive polymers may be hyaluronic acid and its derivatives, chitosan and its derivatives, cellulose and its derivatives and other polymers that have bioadhesive properties known to those skilled in the art.

The water-soluble film forming polymer may be, but is not restricted to, cellulosics and derivatives thereof, poly(vinyl alcohol), poly(ethylene glycol), poly(vinyl) pyrrolidone, poly(ethylene oxide)-poly(propylene oxide)-poly(ethylene oxide) (PEO-PPO-PEO) and other polymers that film-forming and known to those skilled in the art.

Examples of vaccines selected for this application may be, but are not restricted to, the flu vaccine, hepatitis vaccines, DNA vaccines, nucleic acid based vaccines, combination childhood vaccines, all childhood vaccines, vaccines that treat Alzheimer's disease, vaccines that treat multiple sclerosis, vaccines that prevent and treat HPV and vaccines to treat cancer.

Examples of immunomodulating compounds may be, but are not restricted to, therapeutics that treat multiple sclerosis and therapeutics that treat rheumatoid arthritis.

### 03 Concept 2

### Utility as a Therapeutic Composition to Prevent or Treat Mucositis Caused by Radiation Therapy or Cancer Therapy

In one preferred embodiment, the β-Glucan containing therapeutic composition described in this invention can be used to treat mucositis caused by destruction of the mucous membrane as a result of radiation therapy or chemotherapy.

The mucosal surfaces to be treated by the β-Glucan containing therapeutic composition are the oral mucosa, gastro-intestinal mucosa, nasal mucosa, vaginal mucosa, ocular mucosa, and pulmonary mucosa.

Mucositis is a serious and often very painful disorder involving inflammation of the mucous membrane, with the inflammation often accompanied by infection and/or ulceration, typically occurring in immune-compromised persons, typically cancer patients. In general, mucositis is characterized by: (a) an inflammation phase resulting in a cytokine release from the epithelium brought on by damage caused by radiation or chemotherapy, (b) epithelial phase, signaled by atrophy, destruction and ulceration of the mucosal epithelium and a third phase, (c) characterized as the ulcerative phase where lesions form. Due to the ability of β-Glucan to activate immunomodulatory cells to activate/regenerate the immune system and aid in the wound healing process through tissue regeneration, it is rational to include β-Glucan in the therapeutic composition to treat mucositis.

By “treatment” of mucositis, it is meant that the therapeutic composition is efficacious in prevention of the occurrence of mucositis, or enhancement of the rate of wound healing and mucosal tissue re-formation.

### A. Therapy for Oral Mucositis

In one preferred embodiment, the therapeutic composition to treat oral mucositis is comprised of β-Glucan, an antioxidant, an antimicrobial drug, a cytoprotectant and a bioadhesive polymer that would retain the formulation at the site. The β-Glucan may be soluble or particulate, preferably nanoparticulate. Other immunomodulating compounds such as GM-CSF, shown to have effectiveness against mucositis may also be included in the therapeutic composition.

In one preferred embodiment, the β-Glucan containing therapeutic composition to treat oral mucosa may be in the form of a fast-dissolving film.

In another embodiment, the β-Glucan containing therapeutic composition to treat oral mucosa may be in the form of fast-dissolving tablet or troche.

In an alternate embodiment, the β-Glucan containing therapeutic composition to treat oral mucosa may in the form of an extrudable gel, or sprayable viscous liquid.

Preferred embodiments of the therapeutic composition may include, but are not restricted to, inclusion of immunomodulatory agents, topical anesthetics, antiseptics, antibacterial, antifungal and anti-viral agents, cytoprotectants, mucosal cell stimulants and analgesics. Standard of care for oral mucositis include more or more of these compounds to prevent the occurrence of, or reduce the severity of oral mucositis.

Other embodiments of the therapeutic composition may also include compounds that act as “radiation guards”. These may include compounds that have the ability of quench free radicals, such as porphyrins and porphyrin derivatives.

Other examples of compounds that may be included, but are not restricted to, are allupurinol, chlorhexidine and derivatives thereof, povidone-iodine, beta-carotene, vitamin E and oxypentifylline.

In one preferred embodiment, chlorhexidine hydrochloride may be dispersed into the therapeutic composition to enable sustained release of the anti-microbial therapeutic, to enable sustained bioactivity. Chlorhexidine hydrochloride has limited solubility in water and nano- or micro-particles of the therapeutic may provide a prolonged delivery of the drug, if it delivered in a composition that has a long residence time in the oral cavity.

For enhanced performance of the therapeutic composition, it is important that one or more of the components of the therapeutic composition are sufficiently bio-adhesive to promote ready adhesion to mucosal surfaces, thereby promoting retention of the drug adjacent the mucosal surface for effective delivery to the targeted mucosal site. In one preferred embodiment, the biocompatible polymer is bioadhesive, so that when the therapeutic composition is contacted with a mucosal surface, at least a portion of the biocompatible polymer readily adheres to the surface. Preferably, the biocompatible polymer and the drug are closely associated with each other in the therapeutic composition such that when the biocompatible polymer adheres to a surface inside the oral cavity, the drug also adheres to the surface along with the biocompatible polymer. In one embodiment, the therapeutic composition includes, in addition to the biocompatible polymer, a separate bio-adhesive agent that enhances the bio-adhesive properties of the therapeutic composition. The bio-adhesive agent is frequently a second polymer having even greater bio-adhesive properties.

The therapeutic composition can be made with or without thermo-reversible viscosity behavior. By “thermo-reversibility” it is meant that the therapeutic composition exists in a liquid state at low temperatures (refrigerated temperatures) and obtains a “gel-like” consistency at higher temperatures. This property of thermo-reversibility enables administration of a “cold” therapeutic solution that “gels” as the solution contacts the physiological temperature of the mucosal tissues. Administration of a cold solution to inflamed tissue results in a refreshing, soothing effect to the patient. Examples of thermo-reversible polymers in aqueous solution are poly(isopropyl acrylamide), poly(ethylene oxide)-co-poly(propylene oxide)-co-poly(ethylene oxide) and combinations thereof.

As a preferred embodiment, the β-Glucan containing therapeutic composition is preferably administered in the form of a flowable medium with sufficient fluidity for use as a mouthwash that can be swished in the oral cavity.

### B. Treatment of Esophagitis (or Mucositis of the Esophagus)

When treating for esophagitis, the composition will preferably have a very high viscosity as it is swallowed to promote a long residence time in the esophagus and effective coating of mucosal surfaces in the esophagus. For esophageal applications, when the therapeutic composition is administered as a cold flowable medium, the therapeutic composition preferably has reverse-thermal gelation properties.

For targeting mucosal surfaces in the stomach, the therapeutic composition will preferably be in a form so that it can be readily swallowed to coat the mucosal surfaces in the stomach.

Preferred embodiments include those noted for treatment of esophagitis.

### C. Treatment of Nasal Mucositis

For application to nasal mucosal surfaces, it is preferred that the therapeutic composition be sufficiently fluid so as to be nebulizable or otherwise sprayable to generate a nasal spray of the therapeutic composition that can be introduced into the nasal cavity. Preferably, the therapeutic composition is at a refrigerated temperature when sprayed and exhibits reverse-thermal viscosity behavior, so that it undergoes an increase in viscosity as it warms in the nasal cavity, thereby promoting adhesion to mucosal surfaces. For nasal applications, it is preferred that the therapeutic composition have reverse-thermal gelation properties.

### D. Treatment of Rectal or Vaginal Mucositis

For application to rectal or vaginal mucosal surfaces, the therapeutic composition is preferably in the form of a viscous gel when at physiological temperature. The therapeutic composition can be formulated to exhibit reverse-thermal viscosity behavior so that it is administrable in a refrigerated form at a lower viscosity and converts to a higher viscosity form, preferably a gel form, as the therapeutic composition warms following administration.

### 04 Concept 3

### Utility as a Therapeutic Composition to Treat Diseases of the Brain

One of the challenges to treat diseases that are brain-related are achievement of therapeutic levels of the drug in brain tissues. Technical bottlenecks to successful brain delivery is transport of the drug through the blood-brain barrier and short half-lives of the drug in-vivo. Due to the immuno-modulatory nature of the therapies that treat diseases such as Alzheimer's disease, Multiple Sclerosis, Parkinson's Disease and other diseases of the brain, it is rational to utilize beta glucan-containing therapeutic compositions in formats that can enable adequate transport through the blood-brain barrier, enhance the immunological activity and thus, provide enhanced effectiveness and provide protection to the therapeutic to extend its lifetime in-vivo. Another challenge to treatment of multiple sclerosis is in the generation of flu-like symptoms due to the drug therapy (Avonex, Copaxone). This leads to patient non-compliance and discontinuation of the requisite treatment due to discomfort and illness presented as side-effects to the treatment. MS drugs are typically administered intravenously, which leads to distribution of the therapeutic to all tissues. Since most MS drugs are immuno-modulating compounds, targeting the therapeutic composition to the right cells such as the Langerhan's cells in the skin, Peyer's patches in the mucosa, etc. would enhance the effectiveness of drug and result in a smaller dose requisite to generate the desired effect. A smaller requisite dose would likely result in the elimination of side effects of drugs administered for MS.

In a preferred embodiment of the invention, the β-Glucan and drug containing particles would be nano-sized, since nano-sized particles have better transport properties through biological barriers such as mucous membranes.

In another embodiment of the invention, the β-Glucan and drug-containing composition is in the form of a dry film, as described previously in this invention.

In another embodiment of the invention, the β-Glucan and drug-containing composition is in the form of a liquid. In an embodiment, the therapeutic composition discussed herein, would contain of β-Glucan particles dispersed in a biocompatible matrix containing the drug and other components that add functionalities that make the delivery system effective for the application and the route.

The route of delivery can be subcutaneous, wherein the β-Glucan and drug containing biocompatible matrix is injected into the subcutaneous or intramuscular space to achieve sustained delivery of the therapeutic drug. It is to be mentioned here, that the current mode of administration for drugs to treat multiple sclerosis, Alzheimer's disease and Parkinson's disease are given intravenously. Administration of the drug in a sustained manner will result in a lower concentration of drug in the serum, possibly resulting in lesser side effects.

The route of delivery can be in the oral space, whereupon the β-Glucan and drug-containing biocompatible matrix is delivered to the oral mucosa, preferably to the sublingual or buccal tissue, where the tissue type is highly permeable.

The route of delivery can be to the ocular mucosa, nasal mucosa or the pulmonary mucosa, where high vascularization of the tissue leads to efficient absorption of the drug.

The route of delivery can be to the dermal tissue, whereupon the β-Glucan and drug-containing matrix containing components that are permeation enhancing.

The administration of the delivery system to the dermal tissue may be enabled further by use of “delivery aids”. The nature of the delivery aid can be physical/mechanical, or chemical, or combinations thereof. For transdermal delivery of a therapeutic such as Copaxone, the skin can be pre-treated first to remove or disrupt the stratum corneum by micro-dermabrasion, laser micro-ablation, low frequency ultrasound or by chemical delipidation formulations. In one example, the chemical delipidation formulation is comprised of DMSO, triacetin, menthol, benzyl alcohol, polyethylene glycol, DMF, ethyl alcohol, glycerol and derivatives thereof, vitamin E, castor oil, cocamide, quaternary esters, dodecyl sulfate, glycocholic acid, taurocholic acid, cholesterol and derivatives thereof, phosphatidyl ethanolamine, phosphatidic acid, ceramide, stearic acid, oleic acid, PEG-stearate, sorbitan, sorbitols, etc. In one embodiment of a skin pre-treatment delivery aid, the delipidation formulation may be incorporated into a wipe, with or without micro-abrasive microspheres to aid in the delipidation of the stratum corneum.

For nasal delivery of a vaccine, the delivery aid can be in the form of a propellant system that would deliver the vaccine to the nasal epithelium. Conversely, a mucoadhesive can be incorporated into the vaccine delivery system as a delivery aid to enable adherence of the drug to the nasal epithelium prior to uptake. The mucoadhesive can be comprised of mucin, chitosan, polycarbophil, alginates, xanthan gums, Carbopol 971P, gelatin, hyaluronan, hydroxylethyl cellulose, polyacrylic acid, hydroxypropyl cellulose, starch, etc.

In one embodiment of this invention made appropriate for the treatment of brain diseases, the β-Glucan will be particulate, appropriate for the activation of monocytes and macrophages. For efficient cellular uptake, the particles may be nano-sized or micro-sized. In one example of this, the vaccine and particulate β-Glucan will be formulated appropriately in a matrix, suitable for the route (oral, nasal, pulmonary, skin, intramuscular, subcutaneous, etc.) and delivered.

### 05 Other Embodiments that Include Further Description of the Delivery System

In another embodiment of the invention, the β-Glucan will be adsorbed on aluminum hydroxide particles.

In another embodiment of the invention, the vaccine or the antigen can be adsorbed or attached by covalent, ionic or van der waal interactions on the β-Glucan particles (Zymosan), incorporated into an appropriate biocompatible matrix and delivered. The vaccine-containing particles can then be incorporated into a matrix appropriate for the route of administration and delivered.

In another embodiment of this invention, the vaccine or drug will be particulate and will be co-delivered with β-Glucan particles formulated in a matrix suitable for efficient uptake by antigen presenting cells such as monocytes and macrophages. The vaccine can be virus-based, protein-based, or peptide-based, or nucleic acid-based, or small-molecule based. In this capacity, the vaccine particles may be formulated in liposomes, vesicles that are comprised of phospholipids and cholesterol. The vaccine particles can also be in the form of virosomes, comprised of virus-like components.

In another embodiment of this invention, larger particles may be delivered to the site of administration for a higher residence time at the tissue site.

In another embodiment of this invention, the vaccine or drug, β-Glucan and other co-delivery excipients can be formed into a single combination particulate matrix and delivered. The combination particle matrix can be fabricated by emulsion technology, complex coacervation, co-precipitation, etc.

In another embodiment, the β-Glucan polymer may be used as an encapsulant to “encapsulate” drugs and vaccines. The advantage to this approach is that protein and nucleic acid based drugs can be targeted to antigen presenting cells (APCs) such as monocytes and macrophages without being degraded by proteases or nucleases. The β-Glucan polymer thus serves both as a macrophage activator and a protectant. The encapsulating polymer may be β-Glucan alone, or β-Glucan in combination with a biocompatible polymer such as poly (lactide-co-glycolide) (PLG). The co-encapsulating polymer may be charged, such as in PLG with acid end groups or neutral, such as in PLG. The methods of encapsulation of the drug or the vaccine may include water-in-oil—in water emulsifications, o/w/o emulsifications, w/o/o emulsifications, complex coacervation, co-precipitations, etc. Methods of reducing particle size of the particles may include nebulization, then precipitation into a non-solvent, homogenization, sonication and high shear mixing.

In one example of the use of particulate β-Glucan as a vaccine delivery system, the surface of the particles can be coated with compounds that can render the particles cationic, anionic, zwitterionic or neutral. Among these compounds are cetyl triammonium bromide (CTAB), chitosan, polyethyleneimine (linear or branched), CHAPS, stearalkonium chloride, benzalkonium chloride, protamine, poly(lysine), poly(arginine), poly(glutamic acid), taurocholic acid, saponins, glycocholic acid, cholic acid, polyethylene oxide and derivatives thereof, polyethylene oxide and derivatives thereof, PEG-distearoyl phosphatidyl ethanolamine (PEG-DSPE), PEG-dimyristoyl phosphatidyl ethanolamine, PEG-caprate, PEG-stearate, capric acid, stearic acid and derivatives thereof, triglycerides, pluronics, tetronics, glycolipids, etc. The β-Glucan particles can be surface-modified with receptor-binding molecules such as folate, mannose and cholesterol, to enhance cellular binding subsequently followed by uptake.

The physico-chemical properties of the matrix described in this invention may be varied, for the purpose of adding other functionalities to the delivery system. For example, the matrix may be comprised of a muco-adhesive polymer such as alginate or chitosan to aid in the absorption of the drug by enhancement of the gastro-intestinal transit time. In another example, the matrix may be comprised of a composition that aids in permeation of intestinal membranes to enhance drug absorption. To that effect, intestinal permeation agents such as caprates, caprylates, macroglycerides, Vitamin E and derivatives thereof and other like agents, may be included in the matrix composition. In another example, the matrix may contain cell permeants such as magainin, mellitin and other agents that demonstrate cell permeation activity. Other excipients may include other compounds with adjuvant functionalities such as peptidoglycans, glycolipids, lipids, polysaccharides, sugars, toxins, CPG segments of nucleic acids.

In another embodiment of the invention, the vaccine formulation matrix may be developed to meet criteria for transdermal application. Formulations containing β-Glucan in variations of what has been described in this disclosure, may be developed as oils, creams, hydrogels, solid films and viscous liquids capable of delivery to skin. In an example, a solid film can be made by casting a liquid formulation consisting of the vaccine or antigen, solubilized β-Glucan and a polymeric matrix comprised of a polymer such as hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), or pluronics, a plasticising agent, such as glycerol, propylene glycol, or polyethylene glycol, a surfactant such as Tween 20 or Tween 80, and a volatile solvent, such as water, isopropanol, or ethanol. Following casting and subsequent evaporation of the solvent, a solid film is produced. The solid film can be further incorporated on a “band-aid” type dermal patch. Prior to delivery, the solid film can be hydrated with a few drops of water, then applied onto skin as an transcutaneous method of immunization. The patch can be applied to intact skin or skin where the stratum corneum has been disrupted by physical or chemical means, described earlier. Preferably, the hydrogel matrix formulations of the invention comprise water-based hydrogels. Hydrogels are preferred formulations because of their high water content and biocompatibility. As is well known in the art, hydrogels are macromolecular polymeric networks that are swollen in water. Examples of suitable polymeric networks include, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylat-e), poly(n-vinyl pyrolidone), and pluronics. The most preferred polymeric materials are cellulose derivatives. These polymers can be obtained in various grades presenting different average molecular weight and therefore exhibit different rheological properties. The hydrogel formulations of the invention preferably have sufficient surface activity to insure that the formulations exhibit adequate wetting characteristics, which are important for establishing optimum contact between the formulation and the skin. Adequate wetting properties are achieved by incorporating a wetting agent in the hydrogel formulation. Optionally, a wetting agent can also be incorporated in the solid film. The wetting agents include at least one surfactant. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80, and SDS. Preferably, the wetting agents also include polymeric materials or polymers having amphiphilic properties. Examples of the noted polymers include, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics (PEO-PPO-PEO).

The matrix described in this invention can be biodegradable, as well as biocompatible. The matrix may be comprised of segments that may be degradable by enzymes that are present in the body or degradable by the process of hydrolysis by water present in the body. To render a matrix degradable by hydrolysis, the matrix may contain linkages that are subject to scission by water molecules. These linkages may be comprised of esters, amides, carbonates, ester-carbonates, ester-amides. Examples of materials containing these types of linkages are poly(lactide-co-glycolide), poly(trimethylene carbonate), poly(caprolactone) and combinations thereof. Combinations of different types of linkages can be used to modulate the degradation time of these materials by hydrolysis. To render a matrix degradable by enzymatic degradation, the material must contain linkages that are susceptible to enzymolysis. Examples of materials that are susceptible to enzymolysis are hyaluronic acid, chitosan, polylysine, poly(amino acids), etc. Modulating the matrix properties such as hydrophilicity to lipophilicity ratio (HLB), ability of the matrix to absorb water and swell, can affect the diffusive release of drug from the matrix. A matrix that has low absorption of water can result in a slow, sustained release of the drug from the drug delivery matrix. Drug release that is degradation-limited is controlled by the rate of degradation of the matrix, hydrolytic or enzymatic. Other properties of the matrix that can result in controlled release of the drug contained within, are physico-chemical properties of the polymer comprising the matrix. For example, an aqueous polymer solution comprised of poly(ethylene oxide)-poly(propylene oxide)-poly(ethylene oxide) (PEO-PPO-PEO) is a liquid at room temperature and a gel-like solid (semi-solid) at physiological temperature. This enables effective drug loading at low temperature. At body temperature, the gel slows down the diffusion of drug. The gel properties of PEO-PPO-PEO are directly correlated to its concentration in water, thus enabling modulation of drug release from the matrix. Another embodiment of this principle is in the use of crosslinkable polymers as a matrix to deliver drug. A crosslinkable matrix can be pre-loaded with a drug and delivered to the tissue site. As the polymer matrix crosslinks, the covalent network prevents fast release of the drug. The network density of the matrix can be modulated by the molecular weight and concentration of the crosslinking polymers. This provides modulation of the network to control drug release.

What is claimed is:

1. A drug delivery vehicle composition comprising β-Glucan, a water-soluble film-forming polymer, a water-soluble bioadhesive polymer, an antioxidant, and a cytoprotectant.

2. A method for treating cancer comprising the use of a composition as in claim 1, said composition further comprising one or more anti-cancer drugs in an amount effective to treat the cancer, the composition being pre-formed into a pliable film placed under the tongue.

3. A method for treating cancer comprising the use of the composition of claim 2 administered in the form of a pill placed under the tongue.

4. A method for treating cancer comprising the use of the composition of claim 2 that is administered in the form of a dry powder that is inhaled.

5. A method for treating cancer comprising the use of the composition of claim 2 that is administered nasally in the form of a mist.

6. A method for treating cancer comprising the pretreatment of an area of the skin to increase its permeability and the application of the composition of claim 2 formed as a film applied to the pretreated area via skin patch.

7. A method for treating cancer comprising the use of the composition of claim 2 in the form of an enteric-coated pill designed to target specific parts of the gastrointestinal tract.

8. A method for administering a vaccine or other immunomodulatory drug using a composition as in claim 1, said composition further comprising one or more vaccines or immunomodulatory drugs in an amount effective to vaccinate or treat immunological disease, the composition being pre-formed into a pliable film placed under the tongue.

9. A method for administering a vaccine or other immunomodulatory drug comprising the use of the composition of claim 8 in the form of a pill placed under the tongue.

10. A method for administering a vaccine or other immunomodulatory drug comprising the use of the composition of claim 8 in the form of a dry powder that is inhaled.

11. A method for administering a vaccine or other immunomodulatory drug comprising the use of the composition of claim 8 administered nasally in the form of a mist.

12. A method for administering a vaccine or other immunomodulatory drug comprising the pretreatment of an area of the skin to increase its permeability and the application of the composition of claim 8 formed as a film applied to the pretreated area via skin patch.

13. A method for administering a vaccine or other immunomodulatory drug comprising the application onto the ocular mucosa the composition of claim 8 as a fluid.

14. A method for administering a vaccine or other immunomodulatory drug comprising the application onto the ocular mucosa the composition of claim 8 as a film.

15. A method for treating multiple sclerosis comprising the use of a composition as in claim 1, said composition further comprising a one or more drugs in an amount effective to treat multiple sclerosis, the composition being pre-formed into a pliable film placed under the tongue.

16. A method for treating multiple sclerosis comprising the use of the composition of claim 15 administered in the form of a pill placed under the tongue.

17. A method for treating multiple sclerosis comprising the use of the composition of claim 15 administered in the form of a dry powder that is inhaled.

18. A method for treating multiple sclerosis comprising the pretreatment of an area of the skin to increase its permeability and the application of the composition of claim 15 formed as a film applied to the pretreated area via skin patch.

19. A method for treating multiple sclerosis comprising the use of the composition of claim 15 administered nasally in the form of a mist.

20. A method for treating Alzheimer's disease comprising the use of a composition as in claim 1, said composition further comprising one or more drugs in an amount effective to treat Alzheimer's disease, the composition being pre-formed into a pliable film placed under the tongue.

21. A method for treating Alzheimer's disease comprising the use of the composition of claim 20 administered in the form of a pill placed under the tongue.

22. A method for treating Alzheimer's disease comprising the use of the composition in claim 20 administered in the form of a dry powder that is inhaled.

23. A method for treating Alzheimer's disease comprising the use of the composition of claim 20 administered nasally in the form of a mist.

24. A method for treating Alzheimer's disease comprising the pretreatment of an area of the skin to increase its permeability and the application of the composition of claim 20 formed as a film applied to the pretreated area via skin patch.

25. A method for treating Parkinson's disease comprising the use of a composition as in claim 1, said composition further comprising one or more drugs in an amount effective to treat Parkinson's disease, the composition being pre-formed into a pliable film placed under the tongue.

26. A method for treating Parkinson's disease comprising the use of the composition of claim 25 administered as a pill placed under the tongue.

27. A method for treating Parkinson's disease comprising the use of the composition in claim 25 administered as a dry powder that is inhaled.

28. A method for treating Parkinson's disease comprising the use of the composition in claim 25 administered nasally in the form of a mist.

29. A method for treating Parkinson's disease comprising the use of the composition in claim 25 administered as a pill that is swallowed.

30. A method for preventing and treating oral mucositis comprising oral rinsing with the composition of claim 1 in fluid form.

31. A method for preventing oral mucositis comprising the administration of the composition of claim 1 as a fast dissolving lozenge or troche dissolved in the mouth.

32. A method for treating oral mucositis comprising oral rinsing with a composition as in claim 1 in fluid form, said composition further comprising one or more therapeutic drugs in an amount effective to treat oral mucositis.

33. A method for treating oral mucositis comprising the administration of the composition of claim 32 as a fast dissolving lozenge or troche dissolved in the mouth.

34. A method for treating oral mucositis comprising the administration of the composition of claim 32 as a pliable film applied over areas needing treatment.

35. A method for treating oral mucositis comprising the administration of the composition of claim 32 sprayed into the mouth as a fluid.

36. A method for preventing vaginal mucositis comprising the application into the vagina the composition of claim 1 in fluid form.

37. A method for treating vaginal mucositis comprising the application into the vagina the composition of claim 1 in fluid form, said composition further comprising one or more therapeutic drugs in an amount effective to treat vaginal mucositis.

38. A method for preventing rectal mucositis comprising the application into the rectum the composition of claim 1 in fluid form.

39. A method for preventing rectal mucositis comprising the application into the rectum the composition of claim 1 in suppository form.

40. A method for treating rectal mucositis comprising the application into the rectum the composition of claim 1 in fluid form, said composition further comprising one or more therapeutic drugs in an amount effective to treat rectal mucositis.

41. A method for treating rectal mucositis comprising the application into the rectum the composition of claim 40 in suppository form.

42. A method for preventing colonic mucositis comprising the administration into the colon the composition of claim 1 in fluid form.

43. A method for treating colonic mucositis comprising the administration into the colon the composition of claim 1, said composition further comprising one or more therapeutic drugs in an amount effective to treat colonic mucositis.

44. A method for preventing mucositis of the gastrointestinal tract comprising the administration of the composition of claim 1 swallowed as a tablet that targets the different parts of the gastrointestinal tract.

45. A method for treating mucositis of the gastrointestinal tract comprising the administration of the composition of claim 1 swallowed as a tablet that targets the different parts of the gastrointestinal tract, said composition further comprising one or more therapeutic drugs in an amount effective to treat mucositis of the gastrointestinal tract.

46. A method for preventing esophageal mucositis comprising the application of the composition of claim 1 administered as a fluid.

47. A method for treating esophageal mucositis comprising the application of a composition as in claim 1 administered as a fluid, said composition further comprising one or more therapeutic drugs in an amount effective to treat esophageal mucositis.

48. A method for preventing ocular mucositis comprising the application onto the ocular mucosa the composition of claim 1 in fluid form.

49. A method for preventing ocular mucositis comprising the application onto the ocular mucosa the composition of claim 1 as a pliable film.

50. A method for treating ocular mucositis comprising the application onto the ocular mucosa the composition of claim 1 in fluid form, said composition further comprising one or more therapeutic drugs in an amount effective to treat ocular mucositis.

51. A method for preventing nasal mucositis comprising the application into the nasal passages the composition of claim 1 in fluid form.

52. A method for treating nasal mucositis comprising the application into the nasal passages a composition as in claim 1 in fluid form, said composition further comprising one or more therapeutic drugs in an amount effective to treat nasal mucositis.
